(19) United States
(12) Patent Application Publication
(10) Pub. No.: US 2014/0194959 A1
(43) Pub. Date: Jul. 10, 2014

(54) TREATMENT DEVICE FOR THE THERAPEUTIC TEMPERATURE MANAGEMENT OF PARTS OF THE BODY

(71) Applicant: W.E.T. Automotive Systems AG, Odelzhausen (DE)

(72) Inventors: Thomas Fries, Shanghai (CN); Jonathan (Yu) Zhang, Tianjin (CN); Felix (Dali) Zhang, Langfang (CN)

(21) Appl. No.: 14/149,010

(22) Filed: Jan. 7, 2014

(30) Foreign Application Priority Data
Jan. 7, 2013 (DE) 10 2013 000 055.6
Jun. 19, 2013 (DE) 10 2013 010 180.8

(51) Int. Cl. A61F 7/00

(52) U.S. Cl. CPC A61F 7/0085
USPC 607/104

(57) ABSTRACT

The present invention relates to a treatment device for the therapeutic temperature management of parts of the body.

It is provided that the treatment device comprises at least one fluid conveyance device for transporting a cooling fluid.

### BACKGROUND

The present invention relates to a treatment device for the therapeutic temperature management of body parts, in particular for cooling the brain, body surfaces or the blood.

Cooling the brain to a temperature between 33 and 36° C. can be helpful in the treatment of myocardial infarctions, strokes or traumatic brain injuries. However, it is crucial for the success of such a treatment that it be started immediately after the critical event. In addition, a continuous and controlled cooling is necessary. Otherwise, the passive rewarming, which simply exposes the patient to the ambient temperature after the removal of the cooling source, could be excessively rapid. This could trigger intensive blood circulation, which could be critical in injured regions.

A known method consists of cooling the surface of the entire body with cold water. However, this requires intense monitoring and the necessary tub is usually not available at the site of the accident.

Another conventional method consists of a local cooling of the skull or of the carotid arteries with ice. However, in hot climate zones, this is not readily available, and moreover extreme cooling can lead to local tissue damage

In addition, cooling helmets exist, for example, for treating swelling of the brain. However, these systems are inflexible, bulky and also not very efficient.

Internal cooling systems available at this time require invasive methods, in order to position the cooling system within the body (for example, U.S. Pat. No. 8,221,481 B2). However, this is complicated and it requires high level medical training.

### SUMMARY

The present teachings provide a treatment device for the therapeutic temperature management of parts of the body, characterized in that the treatment device comprises at least one fluid conveyance device for transporting a cooling fluid.

### BRIEF DESCRIPTION OF DRAWINGS

FIG. 1 illustrates a side view of a treatment device including an outer housing;

FIG. 2 illustrates a treatment device including a fluid conveyance device; and

FIG. 3 illustrates a film cooling pad applied to a neck.

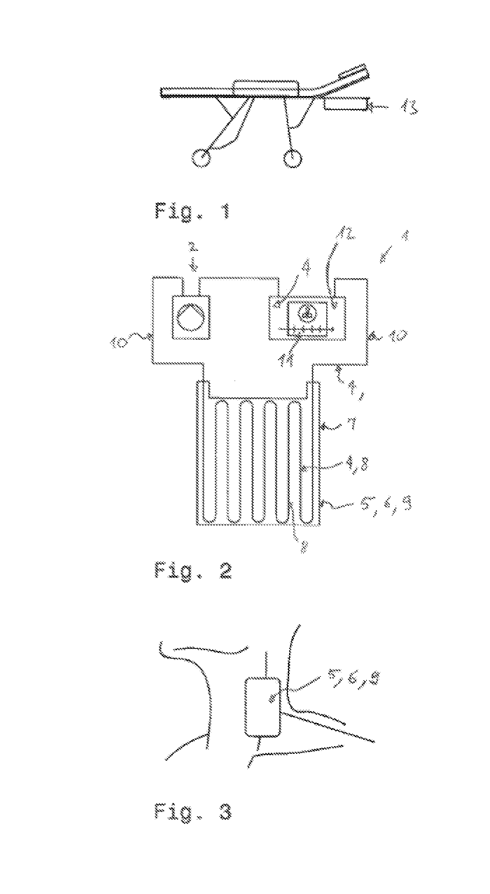

### DESCRIPTION

The invention relates to a treatment device I for the therapeutic temperature management of parts of the body, in particular for cooling the brain. The treatment unit is preferably portable or movable. For example, it is installed in or on a stretcher or at a place where it enables the continuous cooling of the brain from the site of the incident to the hospital.

The treatment unit preferably has at least one fluid conveyance device 2, such as a pump, for transporting or circulating a cooling fluid.

The treatment device preferably has at least one energy source, such as a rechargeable battery system, to allow energy supply during patient transport.

The treatment device preferably has at least one plug for a stationary energy source, such as a socket, for example, in the case where it is used in an ambulance or a operating room.

The treatment device preferably contains or stores at least one cooling fluid 4, for example, a cooling liquid. The fluid should be safe for the patient, even if it is mixed with blood, when it flows out through a leak into a wound. In addition, it should also not solidify at low temperatures.

Good examples are pure water, medicinal glycerol, medicinal alcohols or mixtures thereof,

The treatment device preferably has at least one cooling pad 5 for cooling body surfaces. When such a cooling pad is arranged over blood vessels, it can efficiently cool the blood flowing past in the blood vessel, for example, outside on the neck or over important main arteries.

The cooling pad can have, for example, one or more hook and loop fasteners and it can be applied by means of a hook and loop fastener to the body surface of a patient. Since the cooling pad is at least partially flexible, it fits the size of an adult equally as well as that of a baby. This direct contact and the large contact area increase the cooling effect of the treatment device.

Since the cooling pad, owing to its low weight of less than 50 g in the empty state and less than 500 g, preferably less than 250 g, in the fluid-loaded state, exerts only a very low pressure, it is safe for the patient, even if there is a fracture in the skull of a patient.

The cooling pad is designed preferably as a disposable article and therefore it is manufactured at, least partially from disposable and recyclable materials. As a result, it does not have to be cleaned if the cooling pad is or the tubes are contaminated with the blood of the patient.

Such a cooling pad preferably has one or more partial cooling sections 6. The entire cooling pad can be subdivided into several partial sections that are movable or tiltable relative to one another, in order to make the cooling pad sufficiently soft/flexible to be able to match the curvatures of the body surface.

A cooling pad or a partial cooling section preferably has at least one film 7, in order to form at least one channel. The cooling pad or the partial cooling section preferably has at least two films having the contours of the cooling pad or of the partial cooling section, which cover one another and are attached to each another.

The film is at least partially transparent and produced, for example, from a plastic film, for example, from PU, PP or PE. The transparency makes it possible to monitor the condition of the head of the patient without removing the cooling pad, as a result of which hemorrhages, hyperemia, etc, can be detected rapidly.

The two or more film sections can be glued together or welded in order to form the partial cooling section and one or more channels as a path for the cooling fluid.

A cooling pad or a partial cooling section is preferably provided with at least one fluid channel 8. Such fluid channels can be series connected or parallel connected to the pump.

The treatment device preferably has at least one skull cooler, which is preferably produced at least partially from the plate-like material. It can be configured to be pad-like, bent to a shape, and secured with a hook and loop fastener. It could also be preshaped like a flexible helmet, and adapted with strings to the shape of the head, In this manner, traumatic swellings of the brain can be treated.

The treatment device preferably has at least one alternative or additional neck cooler 9, preferably one for each side of the neck of a patient. The neck cooler is preferably applied over the carotid artery. As a result, arterial blood coming from the heart is supplied in a cooled state to the brain, where it in turn cools the core of the brain. The neck cooler preferably has one or more cooling pads as described above.

The treatment device, in particular the cooling pad, preferably has at least one attachment device. For example, the cooling pad can be applied with hook and loop bands close to the body surface of the patient. This prevents any intermediate insulating layers and establishes a good contact between the body surface and the cooling pad to be cooled. The hook and loop bands can be attached to the cooling pad by means of hook and loop bands, welding or injection molding.

The treatment device preferably has at least one connecting tube 10 for connecting the cooling pads to the pump.

The treatment device preferably has at least one connector for connecting the parts of the system to each other, for example, a textile support, on which at least some of the components (for example, neck cooler, skull cooler and connecting tubes) are arranged, or a string which connects these components to each other. It is also possible to provide at least one valve, in order to control the fluid circulation.

The treatment device preferably has at least one cold generator 11 The latter is preferably a separate module and provided with a temperature control unit. Examples of such cold generators are TED modules (Peltier elements) or condensate/evaporator systems. The cold generator can maintain the cooling fluid at the desired temperature, for example, at approximately 1° C.˜5° C.

The treatment device preferably has at least one fluid reservoir 12 with heat exchanger and heat insulation. The fluid reservoir forms the main container for the cooling fluid. It is attached tightly to the cold surface of the cold generator, so that the fluid contained in it can always be maintained at the desired temperature.

It is possible to manage the temperature of this fluid reservoir beforehand, so as to be able to use the treatment device immediately at the site of the accident. It is also possible to operate the system while bypassing the fluid reservoir. Thus it is possible to cool immediately, even before the fluid reservoir has reached an operating temperature.

The treatment device preferably has at least one user interface, such as keys, buttons, a monitor or display devices. This in combination with the easy use of the cooling devices allows a simple use of the invention even by lay personnel.

The treatment device preferably has at least one outer housing 13 and a holding device, for example, a clamping bracket.

Since the cooling pads are flat, flexible and light, the treatment device is easy to transport and it is easily installed in other systems, such as stretchers or ambulances. It can also be taken out, for example, after taking a patient to a hospital.

### LIST OF REFERENCE NUMERALS

1 Treatment device
2 Fluid conveyance device
Cooling fluid
5 Cooling pad
6 Partial cooling sections
7 Film
8 Fluid channel
9 Neck cooler
10 Connecting tubes
11 Cold generator
12 Fluid reservoir
13 Outer housing

1. A treatment device for the therapeutic temperature management of parts of the body, characterized in that the treatment device comprises at least one fluid conveyance device for transporting a cooling fluid.

2. The treatment device according to claim 1, wherein the treatment device comprises at least one cooling pad for cooling body surfaces.

3. The treatment device according to claim 1, wherein the treatment device comprises at least one cooling fluid, preferably pure water, medicinal glycerol, medicinal alcohol or mixtures thereof.

4. The treatment device according to claim 1, wherein the cooling pad can be applied by means of a hook and loop fastener to the body surface of a patient.

5. The treatment device according to claim 3, wherein the cooling pad is designed as a disposable article.

6. The treatment device according to claim 3, wherein a cooling pad comprises at least one film, in order to form at least one channel.

7. The treatment device according to claim 6 wherein the film is at least partially transparent.

8. The treatment device according to claim 1, wherein the treatment device comprises at least one skull cooler.

9. The treatment device according to claim 1, wherein the treatment device comprises at least one alternative or additional neck cooler.

10. The treatment device according to claim 1, wherein the treatment device comprises at least one cold generator.

11. The treatment device according to claim 2, wherein the treatment device comprises at least one cooling fluid, and the at least one cooling fluid includes pure water, medicinal glycerol, medicinal alcohol, or mixtures thereof.

12. Treatment device according to claim 11, wherein the cooling pad can be applied by means of a hook and loop fastener to the body surface of a patient.

13. The treatment device according to claim 4, wherein the cooling pad is designed as a disposable article.

14. The treatment device according to claim 5, wherein a cooling pad comprises at least one film, in order to form at least one channel,

15. The treatment device according to claim 2, wherein the treatment device comprises at least one skull cooler.

16. The treatment device according to claim 12, wherein the treatment device comprises at least one skull cooler.

17. The treatment device according to claim 14, wherein the treatment device comprises at least one skull cooler.

18. The treatment device according to claim 6, wherein the treatment device comprises at least one alternative or additional neck cooler.

19. The treatment device according to claim 2, wherein the treatment device comprises at least one cold generator.

20. The treatment device according to claim 18, wherein the treatment device comprises at least one cold generator.
